(19) United States
(12) Patent Application Publication
(10) Pub. No.: US 2006/0249146 A1
(43) Pub. Date: Nov. 9, 2006

(54) Auxiliary respiratory device

(76) Inventor: Hsiu-Ching Yu, Lu-Chou city (TW)

Correspondence Address: BACON & THOMAS, PLLC, 625 SLATERS LANE, FOURTH FLOOR, ALEXANDRIA, VA, 22314, US

(21) Appl. No.: 11/120,969

(22) Filed: May 4, 2005

(51) Int. Cl. A61M 16/00

(52) U.S. Cl. 128/202.28; 128/203.11

(57) ABSTRACT

An auxiliary respiratory device includes a hollow body having a first inlet, an outlet in communication with the first inlet and having a first check valve pivotally connected to an inner periphery defining the outlet to allow air to flow only out of the outlet and an air suction hole in communication with the first inlet and having a second check valve pivotally connected to an inner periphery defining the air suction hole to allow air to flow only to the first inlet from the air suction hole.

### BACKGROUND OF THE INVENTION

1. Field of the Invention

The present invention relates to an auxiliary respiratory device to help the person in need to recover ability to breathe.

2. Description of Related Art

Under normal situation, when a person stops breathing due to a number of different reasons, the brain will eventually be damaged due to lack of air. According to statistics, when a person's heart stops beating and cardiopulmonary resuscitation (CPR) is proceeded within four minutes, the brain will be protected from permanent damage. However, when the CPR is performed within four to six minutes, there may be a possibility that the brain will be damaged. And when CPR is performed after six minutes, the brain will definitely be damaged. When CPR is performed after the heart stops for ten minutes, brain death may be the only consequence.

During the golden four minutes, CPR is the best effective measure to restore the patient's heart beating ability. Therefore, it is well known that CPR is able to expand the patient's lung, massage the heart and stimulate the heart to restore the heart beating function. Conventionally, when CPR is applied, it seems that mouth-to-mouth artifical respiration is the only option. That is, the helper's mouth is aligned with the patient's mouth and then the helper breathe in the patient's mouth to help the patient breathe. However, the direct contact between the helper and the patient puts the helper in great danger in that the helper is exposed to different infections from the patient. Sometimes, the patient's vomit during the resuscitation process may endanger the helper's health. In order to avoid such risks, a new respiratory product is introduced to the market. The product has a tubular element to engage with the patient's mouth so that the helper may avoid direct contact with the patient. However, vomit from the patient can still contact the helper because there is nothing in the tubular element to stop the vomit from flowing to the helper's mouth. Later, an improved respiratory having therein a filter or a check valve to stop air or liquid from the patient from flowing to the helper is invented. Drawbacks still exist in such a product in that the filter or the check valve stops most of the air to the patient so that the efficiency is not as good as expected. Furthermore, in order to have large quantity of air, the helper has to move his mouth away from the respiratory device, which is not convenient for the helper. Still, the check valve normally is composed of two slanted valves engaged with each other. The engagement therebetween is not airtight or watertight such that drawbacks from the prior products still exist.

To overcome the aforementioned disadvantages, the present invention aims to provide an improved auxiliary respiratory device to help the patient breathe.

### SUMMARY OF THE INVENTION

The primary objective of the present invention is to provide an improved auxiliary respiratory device having a body provided with a first inlet, an outlet and an air suction hole. The first inlet communicates with the outlet which is in communication with the air suction hole. A first check valve is provided between the first inlet and the outlet. A second check valve is provided between the first inlet and the air suction hole. The firs check valve allows air to flow only from the first inlet to the outlet and the second check valve allows air to flow only from the air suction hole to the first inlet such that the helper is able to have great quantity of air to breathe in to the patient.

Other objects, advantages and novel features of the invention will become more apparent from the following detailed description when taken in conjunction with the accompanying drawings.

### BRIEF DESCRIPTION OF THE DRAWINGS

FIG. 1 is a perspective view of the auxiliary respiratory device of the present invention;

FIG. 2 is an exploded perspective view of the auxiliary respiratory device in FIG. 1;

FIG. 3 is a perspective view in cross section to show the structure of the second check valve;

FIG. 4 is a cross sectional view showing the positions of the firs check valve and the second check valve;

FIG. 5A is a schematic cross sectional view showing the movement of the first check valve;

FIG. 5B is a schematic cross sectional view showing the movement of the second check valve;

FIG. 6 a perspective view of a different embodiment of the auxiliary respiratory device of the present invention;

FIG. 7 is a perspective view of a still different embodiment of the auxiliary respiratory device of the present invention;

FIG. 8 is a perspective view of yet another embodiment of the auxiliary respiratory device of the present invention;

FIG. 9 is a perspective view showing that a belt is attached to the body of the auxiliary respiratory device of the present invention;

FIG. 10 is a perspective view showing that an auxiliary tool to help the patient to open his mouth is attached to the body via a rope; and

FIG. 11 is a perspective view showing that a C-clamp is formed on a side of the body to secure the bellow.

### DETAILED DESCRIPTION OF PREFERRED EMBODIMENT

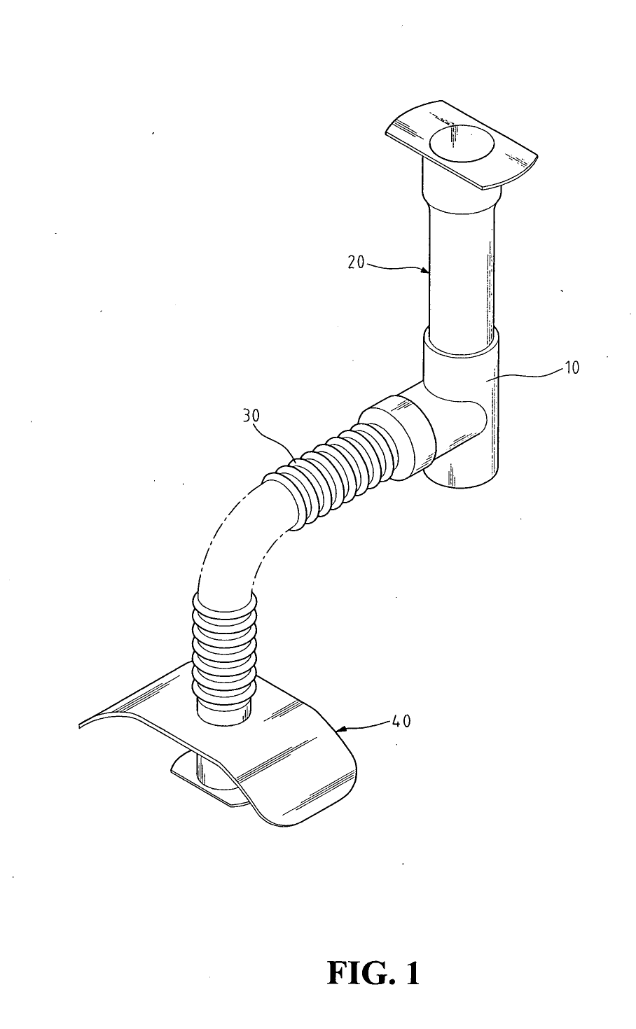

With reference to FIG. 1, the auxiliary respiratory device of the present invention has a hollow body 10, a blowing tube 20 detachably connected to the body 10, a bellow tube 30 detachably connected to the body 10 and a supply tube 40 detachably connected to the bellow tube 30.

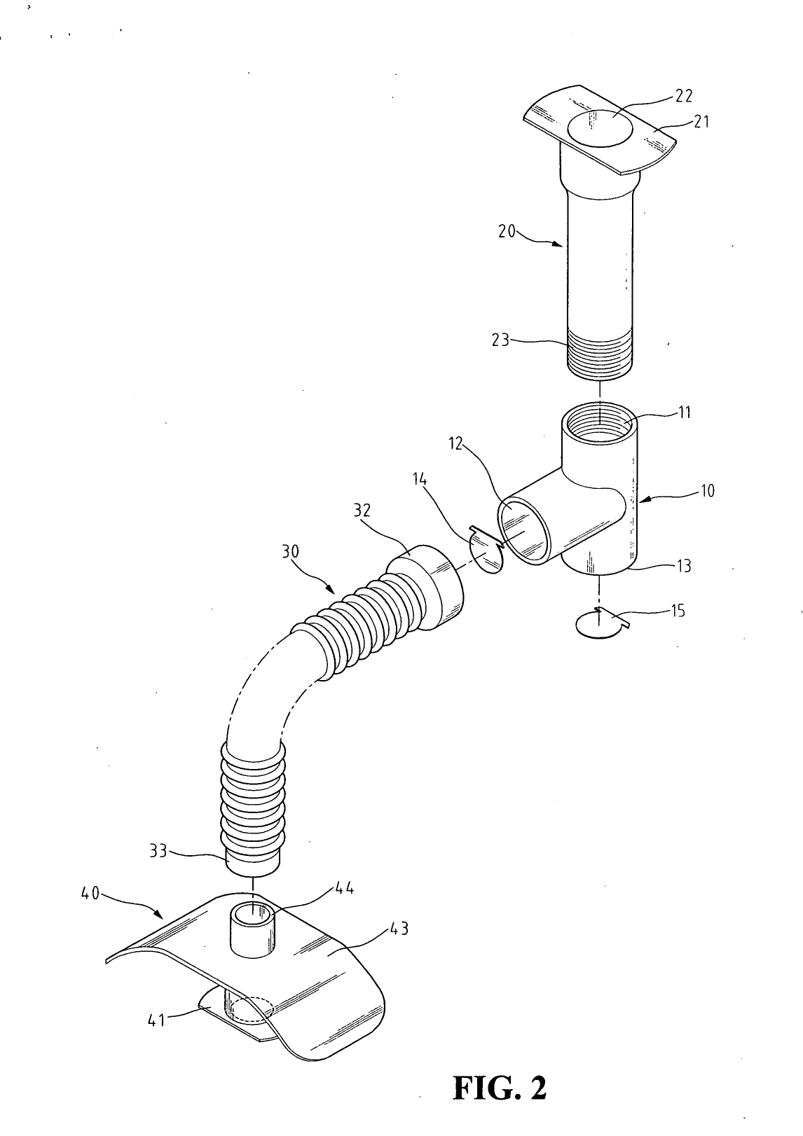

With reference to FIG. 2, it is noted that the hollow body 10 is a three-way tube such that the body 10 has a first inlet 11, an outlet 12 communicating with the first inlet 11 and an air suction hole 13 communicating with both the first inlet 11 and the outlet 12. A first check valve 14 made of a resilient material is provided between the first inlet 11 and the outlet 12. A second check valve 15 is provided between the first inlet 11 and the air suction hole 13. The firs check valve allows air to flow only from the first inlet 11 to the outlet 12 and the second check valve 15 allows air to flow only from the air suction hole 13 to the first inlet 11 such that the helper is able to have great quantity of air to breathe in to the patient.

The blowing tube 20 has two open ends, a fixing plate 21 formed on a periphery defining the top open end thereof, a second inlet 22 defined through the fixing plate 21 and a thread 23 formed on an outer periphery of the bottom open end to correspond to an inner thread (not numbered) formed on an inner periphery defining the first inlet 11 of the body 10. The first fixing plate 21 enables the helper to hold the blowing tube 20 in the mouth so that the helper is able to facilitate the mouth-to-mouth resuscitation. The bellow tube 30 has a third inlet 32 corresponding to and detachably engaged with the outlet 12 of the body 10 and a second outlet 33. The supply tube 40 has two open ends, a second fixing plate 41 formed on a periphery defining a bottom open end of the supply tube 40 and a cover 43 formed on an outer periphery of the supply tube 40. A top open end 44 of the supply tube 40 corresponds to and engages with the second inlet 33 of the bellow tube 30. The second fixing plate 41 is able to extend into the patient's mouth to help straighten the patient's tongue and the cover 43 is able to cover the patient's mouth to avoid possibility of air leakage.

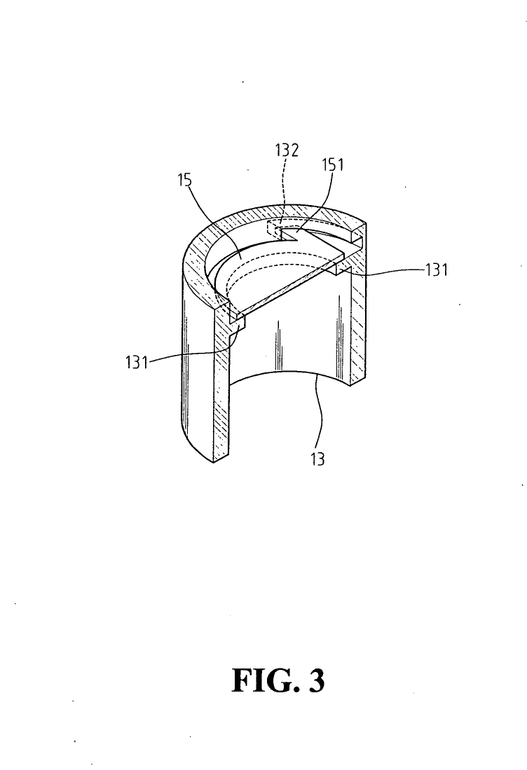

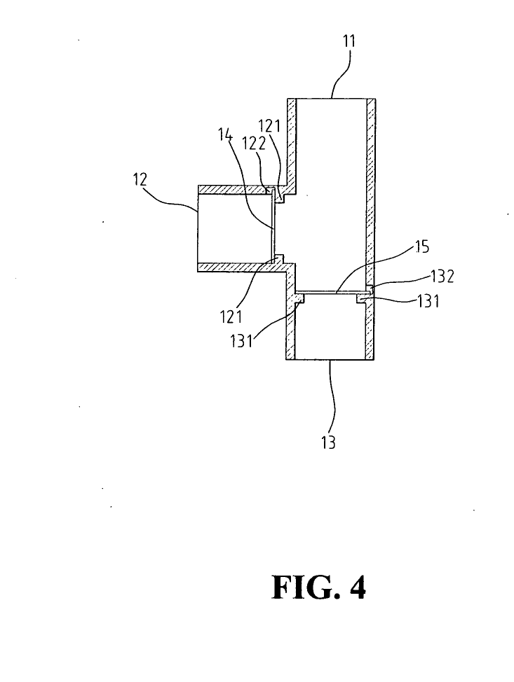

With reference to FIGS. 3 and 4, it is noted that a first groove 132 is defined in an inner periphery of the air suction hole 13 and a first flange 131 is formed on the inner periphery of the air suction hole 13 to be adjacent to the first groove 132. The second check valve 15 has two first ears 151 extending from two opposite points of the second check valve 15 to be received in the first groove 132 and supported by the first flange 131 such that the second check valve 15 is able to pivot away from the air suction hole 13. With reference to FIG. 4, it is noted that a second flange 121 is defined in an inner periphery defining the outlet 12 and a second groove 122 is defined in the inner periphery of the outlet 12 to receive therein the first check valve 14. The first check valve 14 is so configured and received in the second groove 122 that the first check valve 14 is able to pivot toward the outlet 12.

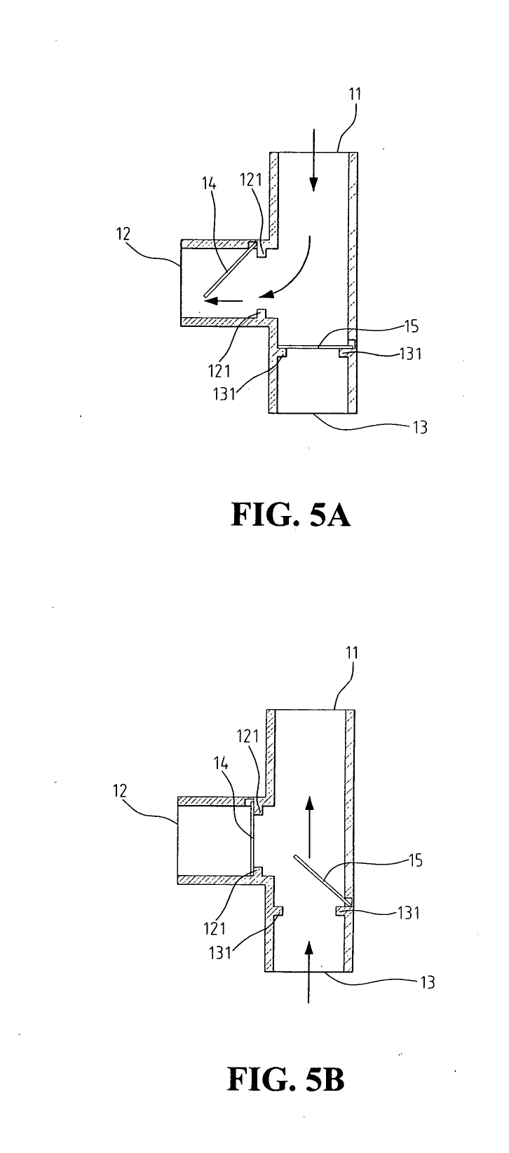

With reference to FIGS. 5A and 5B, when air is coming from the first inlet 11, the second check valve 15 is stopped by the first flange 131 so that the second check valve 15 is not able to pivot. However, due to the position of the second flange 121, the first check valve 14 is able to pivot toward the outlet 12 to allow the air from the first inlet 11 to flow out of the outlet 12. When air is coming from the air suction hole 13, the second check valve 15 is able to pivot toward the first inlet 11 while the first check valve 14 is stopped by the second flange 121 to stop air to flow out of the outlet 12. Therefore, due to the pivotal movement of the second check valve 15, air from the air suction hole 13 is able to flow out of the first inlet 11.

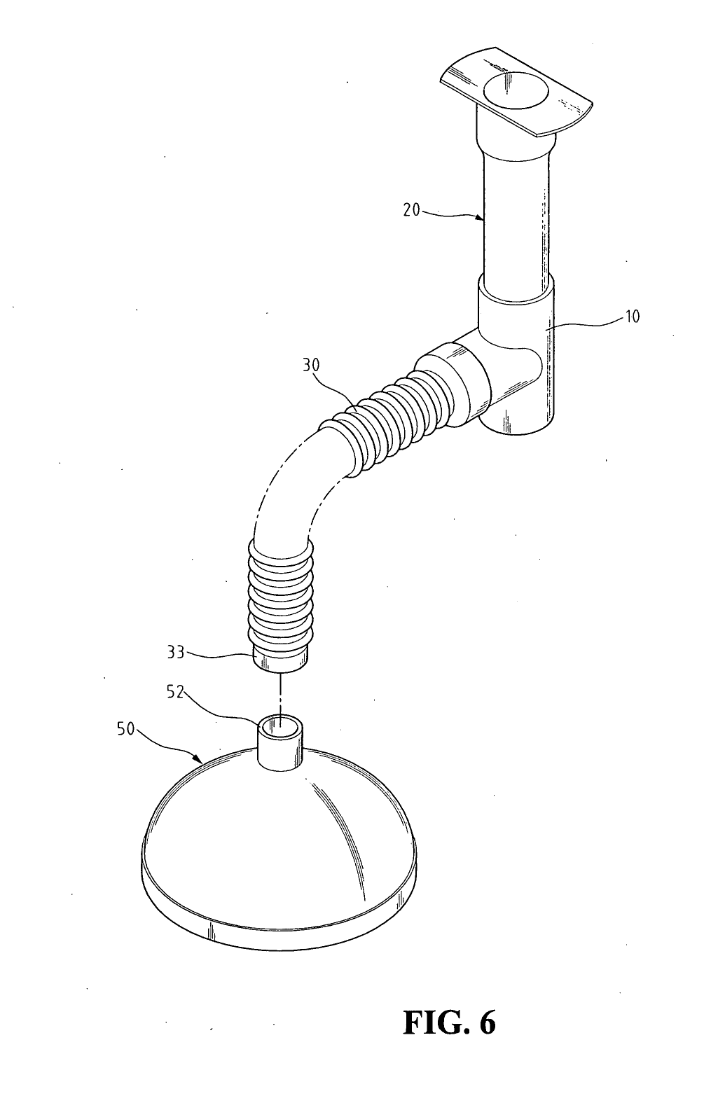

With reference to FIG. 6, the supply tube 40 is now changed to a hollow half circular mask 50 having a tube 52 extending out of the half circular mask 50 and communicating with an interior of the half circular mask 50. The tube 52 is detachably connected to the second inlet 33 of the bellow tube 30. With the provision of the half circular mask 50, the helper is able to proceed the mouth-to-mouth resuscitation with an infant. That is, the half circular mask 50 is able to cover the nose and mouth of the infant.

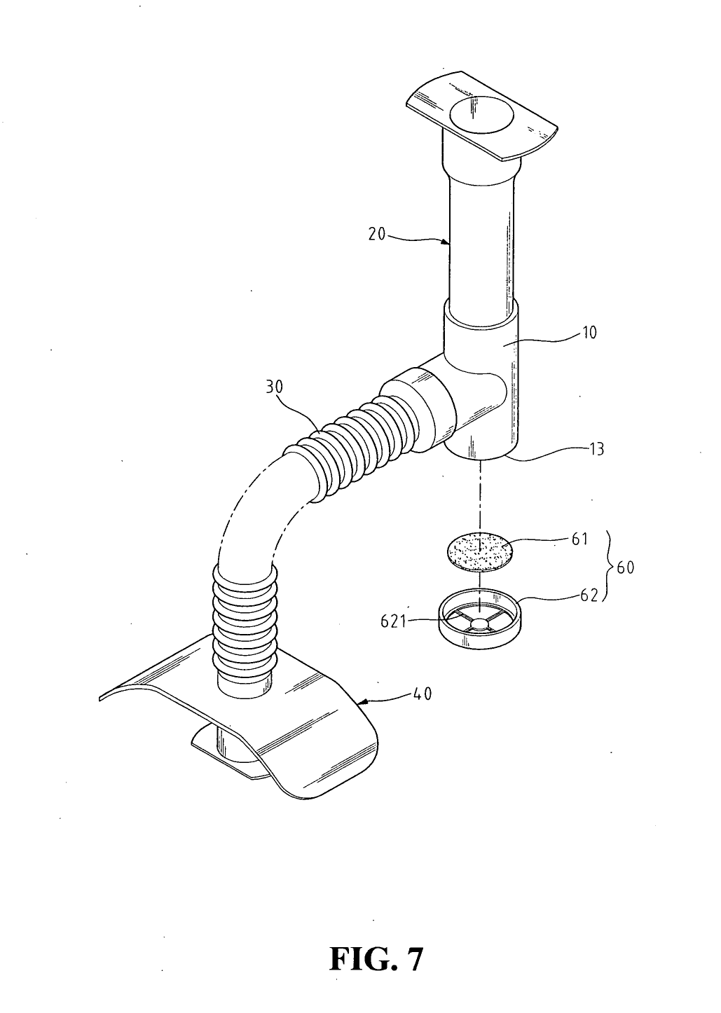

With reference to FIG. 7, a filter device 60 is provided to the auxiliary respiratory device of the present invention. The filer device 60 includes a porous membrane 61 and a cap 62 threadingly connected to the air suction hole 13 of the body 10. The cap 62 has a grill 621 formed on a proximate end thereof and the porous membrane 61 is received in the cap 62 and supported by the grill 621. The porous membrane 61 is able to filter powder or dust in the air so that when the helper is proceeding the mouth-to-mouth resuscitation in a contaminated environment, the filter device 60 is able to prevent the helper from coughing and the resuscitation process will not be hindered.

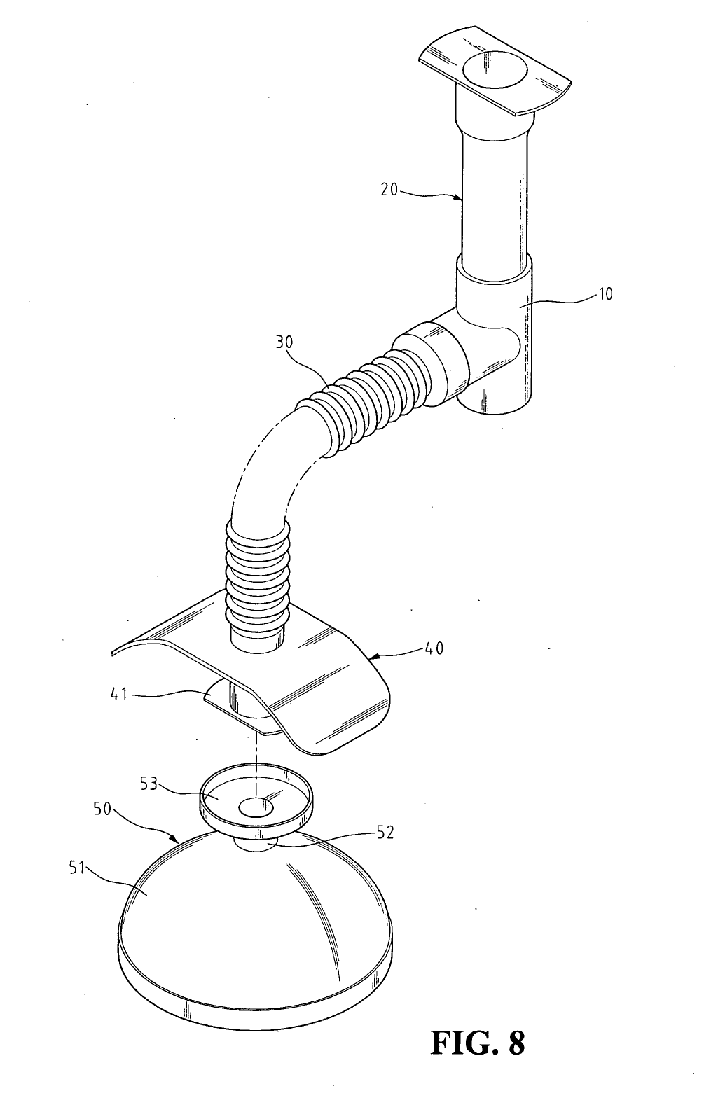

With reference to FIG. 8 and using FIG. 6 for reference, it is noted that the tube 52 is further connected to a disk 53 for connection with the second inlet 33 of the bellow tube 30. The disk 53 has an inner diameter slightly larger than that of the first fixing plate 41 so that the first fixing plate 41 can be securely received in the disk 53.

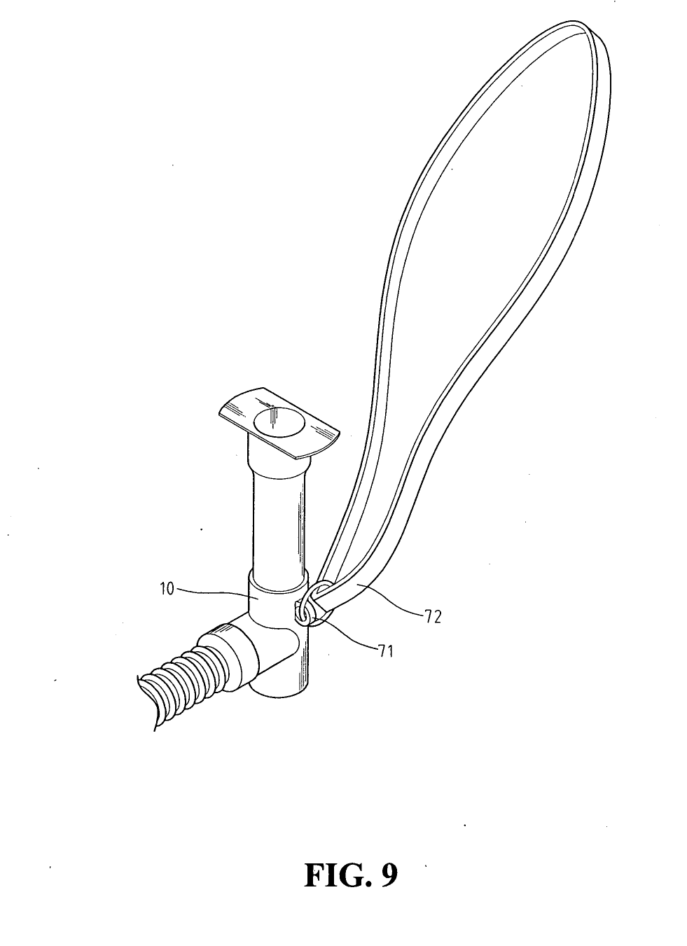

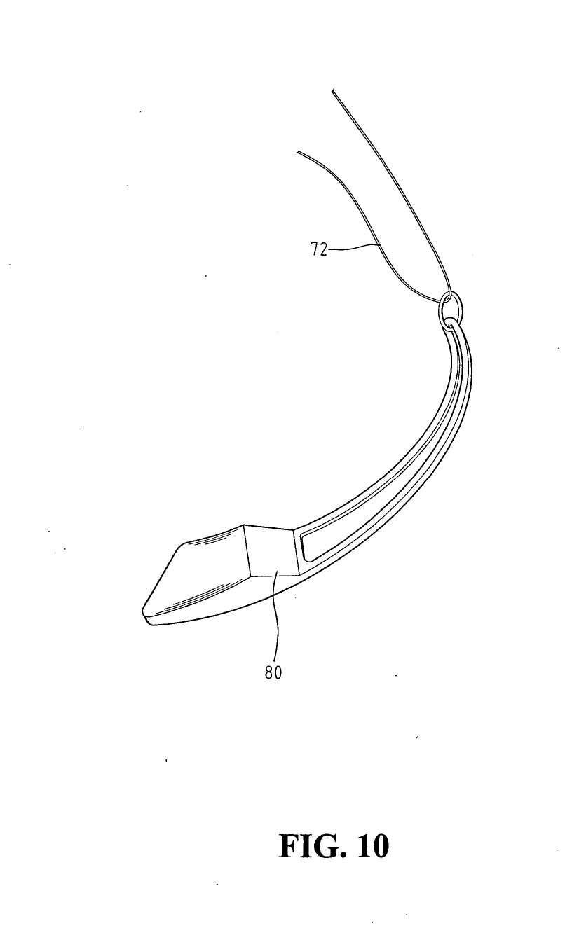

With reference to FIG. 9, a hook 71 is formed on a side of the body 10 to connect to a rope 72 so that the helper is able to hang the auxiliary respiratory device on the neck. With reference to FIG. 10, on the rope 72, an auxiliary tool 80 is also attached for opening the patient's mouth. The auxiliary tool 80 is configured to have a conical appearance such that the helper is able to insert the auxiliary tool 80 into the patient's mouth to help open the patient's mouth for extension of the supply tube 40.

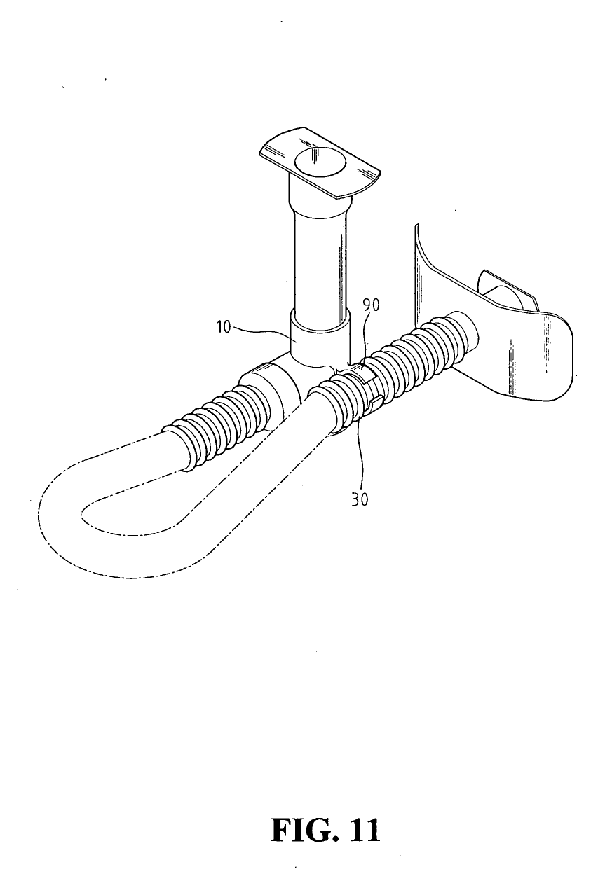

With reference to FIG. 11, a C clamp 90 is formed on a side of the body 10 for clamping the bellow tube 30 so that the auxiliary respiratory device of the present invention is compact when in storage.

Even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. An auxiliary respiratory device comprising:
a hollow body having a first inlet, an outlet in communication with the first inlet and having a first check valve pivotally connected to an inner periphery defining the outlet to allow air to flow only out of the outlet and an air suction hole in communication with the first inlet and having a second check valve pivotally connected to an inner periphery defining the air suction hole to allow air to flow only to the first inlet from the air suction hole.

2. The auxiliary respiratory device as claimed in claim 1 further comprising a supply tube connected to the first inlet of the body via a bellow tube.

3. The auxiliary respiratory device as claimed in claim 2, wherein the supply tube includes a first fixing plate formed on a proximate end of the supply tube and a second fixing plate formed on an outer periphery of the supply tube.

4. The auxiliary respiratory device as claimed in claim 1, wherein a blowing tube is provided to the first inlet of the body, the blowing tube has a second inlet in communication with the first inlet.

5. The auxiliary respiratory device as claimed in claim 2, wherein a blowing tube is provided to the first inlet of the body, the blowing tube has a second inlet in communication with the first inlet.

6. The auxiliary respiratory device as claimed in claim 3, wherein a blowing tube is provided to the first inlet of the body, the blowing tube has a second inlet in communication with the first inlet.

7. The auxiliary respiratory device as claimed in claim 2, wherein a filter device is provided to the air suction hole for filtering out of dust in the air.

8. The auxiliary respiratory device as claimed in claim 3, wherein a filter device is provided to the air suction hole for filtering out of dust in the air.

9. The auxiliary respiratory device as claimed in claim 4, wherein a filter device is provided to the air suction hole for filtering out of dust in the air.

10. The auxiliary respiratory device as claimed in claim 5, wherein a filter device is provided to the air suction hole for filtering out of dust in the air.

11. The auxiliary respiratory device as claimed in claim 6, wherein a filter device is provided to the air suction hole for filtering out of dust in the air.

12. The auxiliary respiratory device as claimed in claim 1, wherein a hollow half circular mask is connected to the outlet via a bellow tube.

13. The auxiliary respiratory device as claimed in claim 12, wherein a blowing tube is provided to the first inlet of the body, the blowing tube has a second inlet in communication with the first inlet.

14. The auxiliary respiratory device as claimed in claim 12, wherein a filter device is provided to the air suction hole for filtering out of dust in the air.

15. The auxiliary respiratory device as claimed in claim 13, wherein a filter device is provided to the air suction hole for filtering out of dust in the air.

16. The auxiliary respiratory device as claimed in claim 11, wherein a hook is formed on a side of the boy to secure a rope.

17. The auxiliary respiratory device as claimed in claim 15, wherein a hook is formed on a side of the boy to secure a rope.

18. The auxiliary respiratory device as claimed in claim 16, wherein a C clamp is formed on a side of the body to secure the bellow tube.

19. The auxiliary respiratory device as claimed in claim 17, wherein a C clamp is formed on a side of the body to secure the bellow tube.

20. The auxiliary respiratory device as claimed in claim 19, wherein an auxiliary tool configured to have a conical appearance is attached to the rope for opening a patient's mouth.
